(19) United States
(12) Patent Application Publication
(10) Pub. No.: US 2014/0088573 A1
(43) Pub. Date: Mar. 27, 2014

(54) PROJECTOR DEVICE, AND MEDICAL DEVICE COMPRISING THE PROJECTOR DEVICE

(71) Applicant: EYESIGHT & VISION GMBH, Nuemberg (DE)

(72) Inventors: Kurt Heiberger, Nuernberg (DE); Andreas Schnalke, Nuernberg (DE)

(73) Assignee: EYESIGHT & VISION GMBH, Nuernberg (DE)

(21) Appl. No.: 14/003,076

(22) PCT Filed: Mar. 4, 2011
(86) PCT No.: PCT/EP2011/053263

§ 371 Date: Nov. 8, 2013

(51) Int. Cl. A61B 3/00; A61F 9/008; A61B 3/10

(52) U.S. Cl. CPC A61B 3/0091; A61B 3/0008; A61B 3/1015; A61F 9/008
USPC 606/4; 351/200; 351/237; 351/211

(57) ABSTRACT

The aim of the present invention is to provide a projector device and a medical device with said projector device, said devices facilitating an improved projection of a two-dimensional pattern onto a plane in an optical body, in particular in an eye.

For this purpose, the invention proposes a projector device for projecting a two-dimensional pattern on a plane in an optical body, in particular in an eye, with a light source which is designed to emit a light beam, with a deflecting device which is designed to deflect the light beam in order to generate the two-dimensional pattern, with an optical module which is arranged between the light source and the plane, and with a control device which is designed to control the deflecting device such that the two-dimensional pattern is formed on the plane, wherein the optical module has at least two regions with different focal lengths through which said light beam can be guided in an alternating or consecutive manner by means of the deflecting device, and wherein the control device is designed to control the deflecting device and the light source such that the light beam is guided through the at least two regions in order to compensate for position-resolved aberrations of the optical body and to project the two-dimensional pattern on the plane such that optical aberrations are corrected.

The invention relates to a projector device for projecting a two-dimensional pattern on a plane in an optical body, in particular in an eye, with a light source which is designed to emit a light beam, with a deflecting device which is designed to deflect the light beam in order to generate the two-dimensional pattern, with an optical module which is arranged between the light source and the plane, and with a control device which is designed to control the deflecting device such that the two-dimensional pattern is formed on the plane. The invention also relates to a medical device with the projector device.

Various methods for the diagnosis and correction of visual detects in the human eye have been established on ophthalmology. In order to correct ametropia it is quite common, for example, to change the surface shape of the cornea through laser ablation of the surface of the cornea. Other treatment methods involve, for instance, the so-called “welding” of the retina to stop its detachment. Many treatment methods in ophthalmology are performed on an outpatient basis where in particular the eye of the patient is prepared only to a small extent or not at all, so that the patient will be able to change, for example, the viewing direction and also the accommodation of the lens of the eye during treatment.

Since such an uncontrolled change of the viewing direction and/or accommodation is not desirable, the suggestion has been made, for example in publication DE102006005473A1, to display an eye test character or accommodation target in the eye of the patient and to instruct the patient to fixate on the object shown by the eye test character or accommodation target. In this way the patient can be forced to keep the viewing direction constant and reduce the accommodation of the eye, which is hard to control, to a minimum. The above-mentioned publication proposed to create the accommodation target by means of a laser beam which is guided via a two-dimensional scanner, said scanner deflecting the light beam and thereby using the laser beam to create a two-dimensional pattern on the retina of the eye. This publication also proposes to compensate for any distortion of the image by means of an interplay between the scanner and a deactivation and activation of the laser beam source.

The aim of the present invention is to provide a projector device and a medical device with said projector device, said devices facilitating an improved projection of a two-dimensional pattern onto a plane in an optical body, in particular in an eye.

This problem is solved by a projector device with the features of claim 1 and by a medical device with the features of claim 17. Preferred or advantageous embodiments of the invention are derived from the subclaims, the following description and the accompanying figures.

The invention thus relates to a projector device for projecting a two-dimensional pattern on a plane, wherein the two-dimensional pattern may be designed as a geometric figure, a letter, a symbol or a numeral but also as an image, partial image, icon etc.

The plane onto which the two-dimensional pattern is projected is arranged in an optical body, in particular in a (human) eye. In particular, the plane in the eye is defined by the retina of the eye, namely by the so-called macula of the retina which is the region of the retina responsible for visual acuity. In this regard, the plane is an auxiliary construct which needs to be associated with an extension of the surface of the macula.

The projector device comprises a light source which is designed to emit a light beam. The light beam can be implemented, in particular, as a laser beam. The light beam may also comprise a time sequence of light beams or laser beams of different colors or wavelengths or a superimposition of light beams or laser beams of different wavelengths. Particularly preferably the light beam is designed as being smaller than 3 mm with regard to the beam diameter, more preferably smaller than 1 mm and in particular smaller than 0.5 mm so that it can be guided through the projector device with exact positioning.

A deflecting device is designed to deflect the light beam in order to create the two-dimensional pattern. When the two-dimensional pattern is created, it is written, for example, on the plane, meaning that it is displayed, for example, by a succession of rows or columns. Instead of rows or columns, other tracking paths may be implemented for the two-dimensional pattern.

The deflecting device can in principle be designed as any scanner device, such as a moving prism, a moving lens etc. Preferably, however, the deflecting device is designed as a mirror scanner device which makes it possible to deflect the light be are at a deflection angle into two independent directions. The mirror scanner device is thus designed such that it can be controlled in two dimensions. In order to save installation space and allow for high dynamics, it is particularly preferable that the mirror scanner device is designed as a scanning micromirror device, wherein the micromirror has a free diameter of less than 7 mm, more preferably of less than 3 mm and in particular of less than 2 mm.

The projector device comprises at least one optical module which is arranged in the beam path of the light beam between the light source and the plane. In particular, the optical module is designed as a beam-guiding or beam-shaping element for the light beam. The optical module can form a single piece or be built of several pieces.

A control device controls the deflecting device and, if necessary, the light source such that the two-dimensional pattern is formed on the plane, and in particular such that said pattern is written or displayed via scanning. For this purpose, the control device comprises, for example, a sequential program or is designed in any other way, due to its programming and/or wiring, to control the deflecting device accordingly.

In accordance with the invention, it is proposed that the optical module should have at least two regions with different focal lengths through which said light beam can be guided in an alternating or consecutive manner by means of the deflecting device. The at least two regions can thus be arranged, with respect to the light beam's direction of propagation, either adjacent to each other or one after the other without overlapping. The deflecting device thus makes it possible to direct the light beam selectively onto one or the at least two regions. If, for example, light beams which are aligned parallel to the optical axis of the optical module are guided through the at least two regions then said light beams will be projected onto two different focal points. In particular, the light beams will not meet at a common focal point. The at least two regions could form, for example, at least two different focal points. Optionally, the at least two regions may have different optical axes. Particularly preferably, the optical module is designed as being changing with respect to refractive power or focal length in its direction of rotation or radial direction, in particular discontinuously changing or non-differentiably changing.

It is also proposed that the control device controls the deflecting device and the light source such that the light beam is guided through the at least two regions, in particular in a selective or alternating or consecutive manner, in order to compensate for position-resolved optical aberrations in the beam path of the light beam, in particular for position-resolved optical aberrations of the optical body, specifically of the eye, in a position-resolved way said to project the two-dimensional pattern on the plane so that optical aberrations are corrected.

It is an idea of the invention that both diagnosis and treatment are advantageously affected if the two-dimensional pattern is displayed undistorted and in sharp focus on the plane, in particular on the retina, specifically on the macula of the retina. This is made difficult, however, by the fact that optical aberrations accumulate in the beam path of the light beam. The optical aberrations can be caused either by the optical system of the projector device or by the optical body, in particular the eye. Without any further action taken, the optical aberrations can cause the two-dimensional pattern to appear out of focus or distorted on the plane.

To compensate for this effect and correct the optical aberrations, it is proposed within the scope of the invention that the light beam serving to project the two-dimensional pattern is guided selectively onto the at least two regions with different focal lengths. This makes it possible to use the control device to select a suitable deflection angle in the deflecting device for any desired image pixel of the two-dimensional pattern on the plane and thus to select a suitable region of the optical module which will project the image pixel enter consideration of the optical aberrations onto the desired position on the plane. If this same procedure is followed for each image pixel of the two-dimensional pattern, an image of the two-dimensional pattern where the optical aberrations are corrected will be displayed on the plane, in particular on the retina, specifically on the macula.

Looking at a very simple embodiment, the optical module would be designed, for example, as a lens module with regions in the form of several circular segments, for example four circular segments, wherein each of the circular segments has a different focal length. If the optical aberration to be corrected for the two-dimensional pattern is, for example, a defocus, the light beam will be guided through the circular segment with a focal length that is most suitable to correct the defocus. However, it is also possible that different regions of the two-dimensional pattern exhibit different optical aberrations. In that case, a first partial region of the two-dimensional pattern will be displayed by projecting the light beam through a first circular segment with a first focal length and a further partial region of the two-dimensional pattern will be displayed by projecting the light beam through a second circular segment with a second focal length. Using this systematic approach, each image pixel of the two-dimensional pattern can be displayed with a light beam which is guided through the region with the focal length that corrects the optical aberrations, and in particular the one that corrects them in an optimal way. The different regions with different focal lengths do not have to be distributed over circular segments, as described in the exemplary embodiment, but can just as well be arranged in the optical module in any other way, as will be shown below by way of different possible embodiments of the invention.

The advantage of the present invention is that two-dimensional patterns can be displayed on a plane such that optical aberrations are corrected while keeping the systems technological expense to a minimum. When implementing the chosen way of displaying the two-dimensional pattern via a writing or scanning method, the deflecting device is already present and the control device must, also be provided due to the underlying principle. The only additional component is the optical module which is, however, designed as a static optical module and thus does not significantly increase true complexity of the mechanical design of the projector device. Implementing the invention thus increases the production costs of the projector device only by the costs of providing a mount and the optical module itself, which is controlled via software.

In a particularly preferred embodiment of the invention, the two-dimensional pattern is designed as an accommodation target or eye test character for the eye. When an accommodation target is used, an image of an object etc. is projected as a two-dimensional pattern, on the macula of the patient and the patient is instructed to fixate on this image. This accommodation target is designed such that both the viewing direction and the accommodation of the lens can be selectively adjusted due to the patient's fixation. In a preferred embodiment, the accommodation target is displayed such that the lens of the eye accommodates to infinity, thereby entering a relaxed condition. This is the state in which the eye is best prepared for examination or treatment.

In a preferred further form of the invention, the light source is designed as a laser beam source which facilitates the emission of several laser hearts of different colors such that the two-dimensional pattern appears multicolored. The use of a multicolored two-dimensional pattern, optionally one with changing colors, substantially improves patient comfort during treatment. Effects such as the feeling of having to “stare” continuously at a red dot that seems to “burn” into the retire are eliminated by having the whole eye test character or partial regions thereof change their color. It is also possible to change the shape or the displayed pattern of the accommodation target so that the outline of the two-dimensional pattern constantly changes, as well.

In another embodiment of the invention which is primarily intended for measurements of the eye, the light beam is in visible. In particular, the two-dimensional pattern forms a measurement pattern. By scanning the eye with an invisible light beam, it is possible to scan the surface of the eye, to evaluate backscattering or reflections with suitable sensor devices such as wavefront measuring instruments and to determine optical aberrations of the eye. The accuracy of these measurements increases if the distortion of the measurement pattern displayed on the retina is minimized, so this embodiment of the invention increases measurement accuracy. It is also possible to design the light source such that visible, in particular single-colored or multicolored, light beams and invisible light beams are projected either alternately or simultaneously on the plane in order to use, for example, the visible light beams to create an accommodation target for fixating the viewing direction and the accommodation of the lens and the invisible light beam to measure the surface of the eye.

In a preferred further form of the invention, the control device is designed such that it adjusts the deflecting device and the light source in order to facilitate a lateral shifting of the two-dimensional pattern on the plane, in particular on the retina, specifically on the macula of the retina.

Lateral shifting can provide, for example, a stimulus for the patient to change the viewing direction, wherein by shifting the two-dimensional pattern and optionally by instructing the patient to fixate on the object shown by the two-dimensional pattern, the patient is prompted to change the viewing direction. Alternatively, a movement of the two-dimensional pattern, in particular the measurement pattern, can be used for a selective measurement of the eye. The projector device may comprise, for example, an operating unit, said operating unit allowing for manual input which causes a movement of the two-dimensional pattern on the plane.

In a preferred constructional embodiment of the invention, the deflecting device is controlled with always the same deflection pattern or with any irregular deflection pattern which does not follow the outline of the symbol formed by the two-dimensional pattern. It is possible, for example, to control the deflecting device in a resonance mode so that the light beam is always deflected using the same sequence of deflection angles. This embodiment has the advantage that very compact scanning micromirrors which operate in resonance mode can be used. Such scanning micromirrors have resonant frequencies between 100 Hz and 150 kHz or more so that the two-dimensional pattern can be refreshed several times per second, in particular more than 30 times per second, which prevents the patient from experiencing a flickering effect. The outline of the two-dimensional pattern and the selection of the regions, on the other hand, are controlled by switching the light source on and off. Alternatively or additionally, the deflecting device is controlled such that the surface area being scanned on. the plane is always complete or continuous and that the accommodation target, eye test character and/or measurement pattern is achieved by selectively switching the light source on and off.

In a possible constructional embodiment, the deflecting device consists of two one-dimensional (meaning they deflect in only one direction) scanning mirror devices as 1D deflecting devices arranged separately to each other, which together facilitate a two-dimensional deflection of the light beam. In particular, at least one optical element is arranged in a beam path between the two 1D deflecting devices. By using separate 1D deflecting devices it is possible to mechanically decouple the two directions of deflection, thus reducing disturbance factors during operation of the projector device.

In a constructional implementation of the invention, the different, regions of the optical module with their different focal lengths may be arranged separately to each other and/or the regions of the optical module with their different focal lengths may be connected via transition zones in which the focal lengths are preferably steadily changing.

As an optional supplement, the optical module has a measuring point region with a focal length chosen such that a focal point is focused on the cornea of the eye as a distance measuring point. Said distance measuring point can be recorded with any camera and analyzed with regard to its diameter. The distance measuring point can be used for adjusting or checking the correct distance between the projector device and the eye. The projector device will be, for example, automatically or manually manipulated to adjust the distance such that the diameter is minimized, thus setting the desired distance.

In a first possible embodiment of the optical module, said optical module is designed as a refractive and/or transmitting optical module. In this embodiment, the optical module preferably resembles a lens, wherein the different regions may be arranged, for example, as circular segments or as circular icings with different diameters. These may be, for example, so-called gradient-index lenses (GRIN) where the focal length changes in a radial direction. In this embodiment, the deflection angle of the deflecting device selects a radial region of the optical module for each image pixel of the optical pattern on the plane, which leads to a display where the optical aberrations are corrected.

In another embodiment or the invention, the optical module can be a diffractive optical module, wherein the different focal lengths are achieved through interference patterns on or in the optical module. Today such diffractive optical elements (DOE) are inexpensive to manufacture industrially, so that a DOE of this type can be mass-produced cheaply after the design stage. In particular, these could be portions of zone plates or Fresnel lenses.

It is also possible to design the optical module as a reflective optical module wherein the regions with different focal lengths result from different reflection regions of the optical module.

In yet another embodiment of the invention, the optical module may be designed as a holographic optical module.

In a possible further form of the invention, the projector device projects a two-dimensional pattern into each eye of the patient, so that both eyes can be provided with a two-dimensional pattern without manipulating the projector device. Thus it is possible, in particular, to measure both eyes simultaneously or in parallel. This further form improves patient comfort during treatment and decreases the chair time required for the examination. Particularly preferably, both two-dimensional patterns can be designed such that the patient is provided with a 3D image.

A further object of the invention relates to a medical device with the projector device of any one of the preceding claims, wherein the medical device is preferably designed as a wavefront measuring instrument, a cataract microscope or a treatment laser system.

In a preferred further form of the invention, the medical device comprises a wavefront sensor for determining the position-resolved optical aberrations of the optical body, in particular the eye. Particularly preferably, the wavefront sensor uses the backscattering or reflections of the two-dimensional pattern as input signals. The wavefront measuring instrument and the projector device may also be designed as a single control circuit or regulator circuit where, in the case of the regulator circuit design, the optical aberrations that have been measured are corrected by controlling the projector device so that after a few regulation cycles, a two-dimensional pattern where the optical aberrations are corrected is displayed on the plane.

In a particularly simple and thus cost-effective embodiment of the invention, no layer thickness measurement of the eye is carried out. Usually there are several layer boundaries present in the eye:

external side—cornea
cornea—anterior chamber
anterior chamber—lens
lens—vitreous body

By measuring back reflections from two different layer boundaries, it is generally possible to calculate the layer thickness between these layer boundaries. In order to implement a simple and cost-effective design, however, it is prefer red that the medical device evaluates only back reflections from at most one of the layer boundaries mentioned.

In a particularly preferable embodiment of the invention, the medical device has a treatment laser, wherein the treatment laser is guided along the same axis as the light beam so that the beam of the treatment laser, just like the light beam, is guided onto the plane, in particular onto the retina, such that optical aberrations are corrected, the treatment laser being used, for example, to treat the retina, in particular to “weld” it. This systems technological design therefore improves the quality of the treatment.

Other features, advantages and effects of the present invention are derived from the following description of preferred embodiments of the invention and the accompanying figures. The figures show:

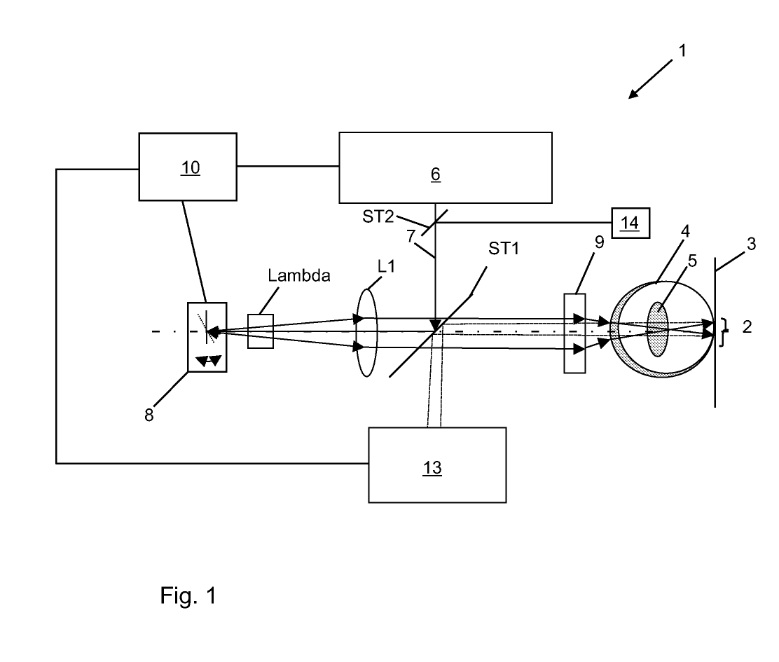

FIG. 1 a schematic block diagram of a projector device as a first embodiment of the invention;

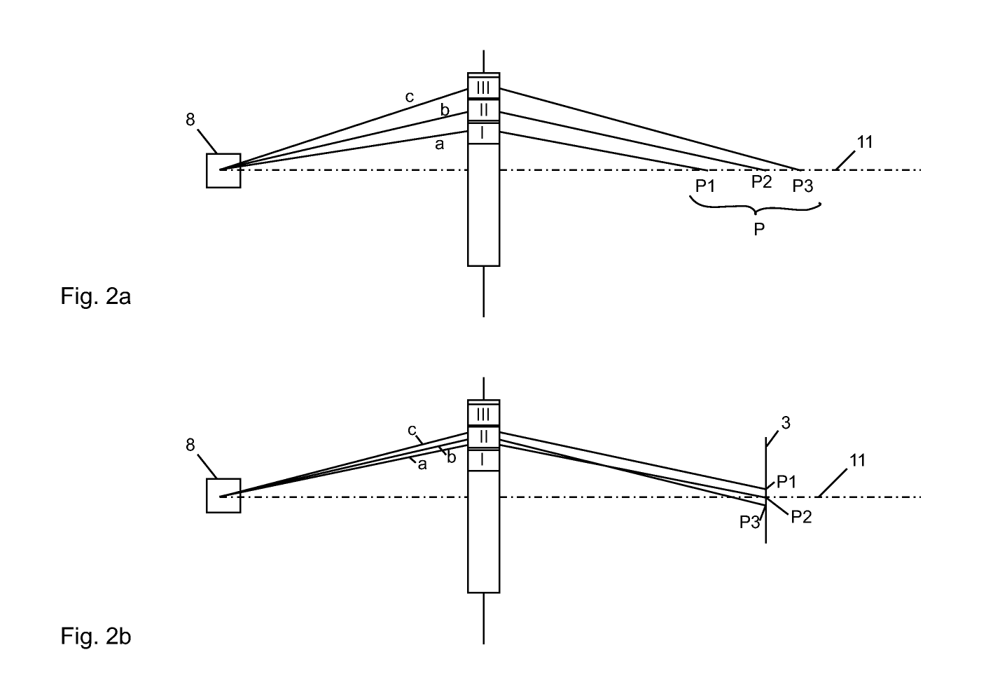

FIG. 2 a, b schematic beam paths illustrating how the projector device works;

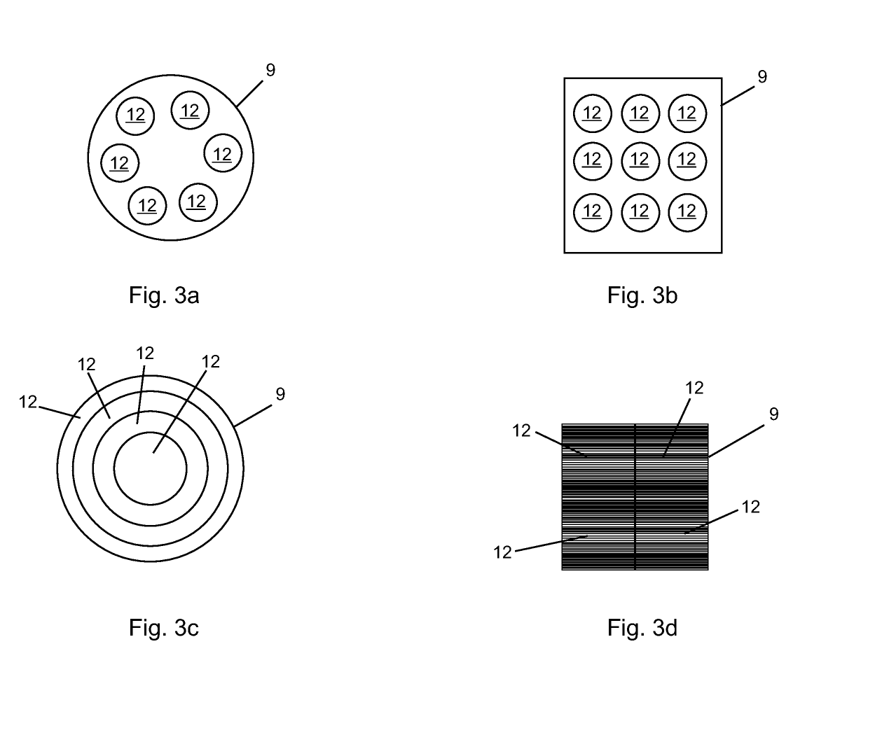

FIG. 3 a, b, c, d different embodiments of an optical module in the projector device of FIG. 1;

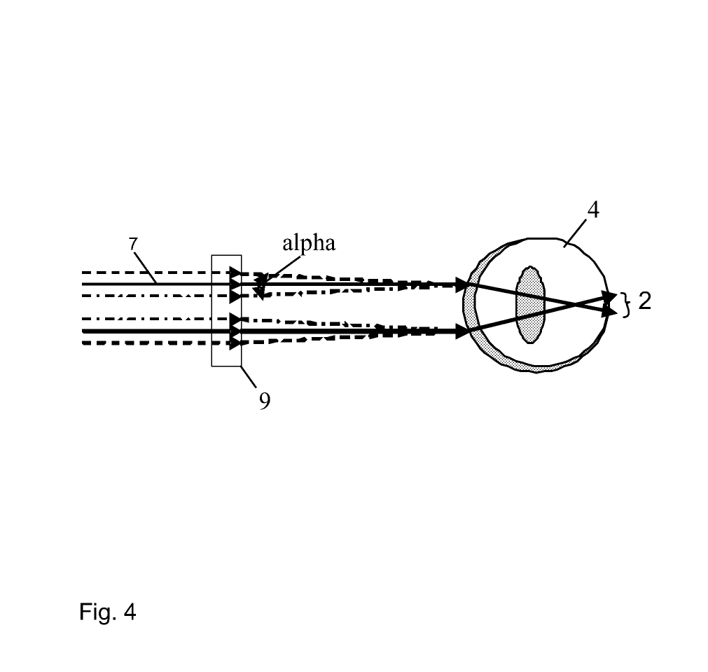

FIG. 4 a detail section of FIG. 1 in a possible embodiment of the invention;

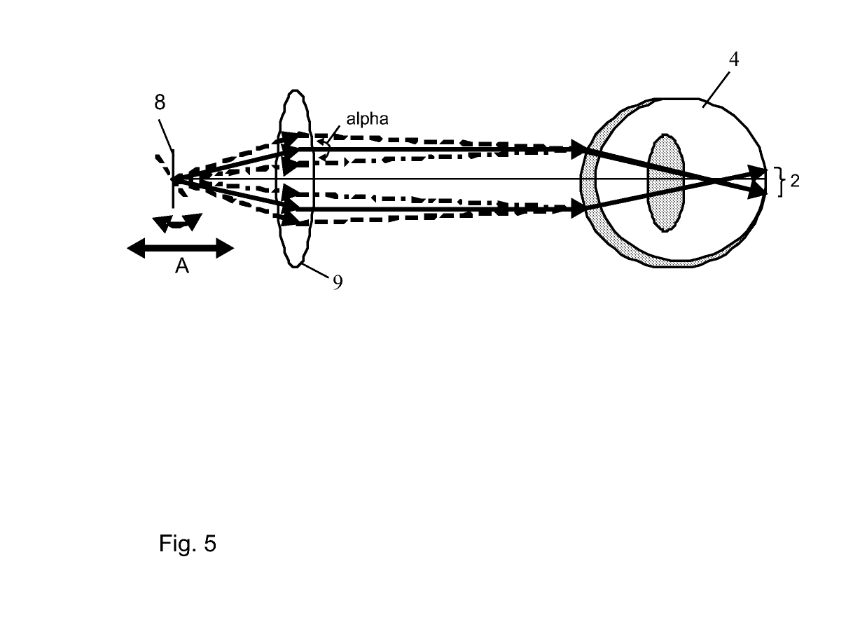

FIG. 5 a similar detail section as in FIG. 4 in another possible embodiment of the invention;

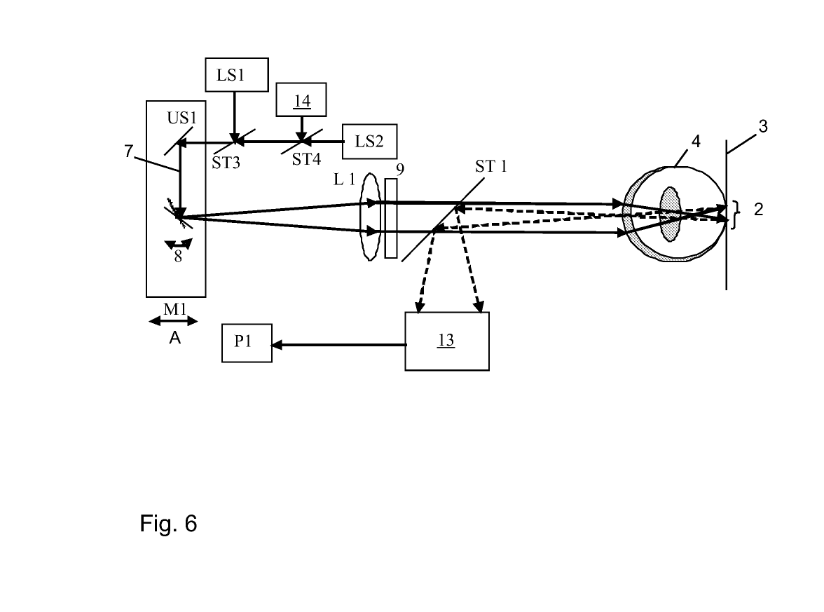

FIG. 6 a schematic block diagram of a projector device as a accent embodiment of the invention;

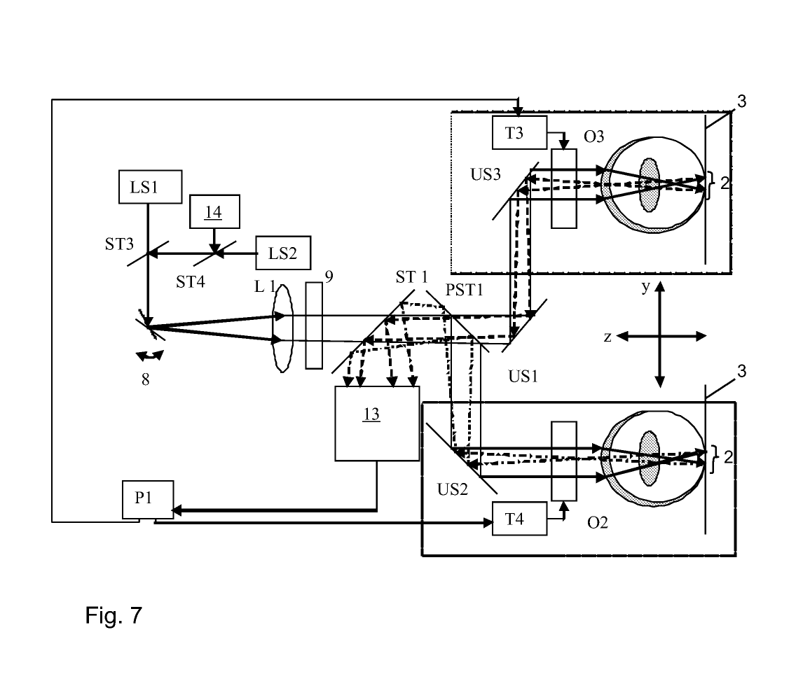

FIG. 7 a schematic block diagram of a projector device as a third embodiment of the invention;

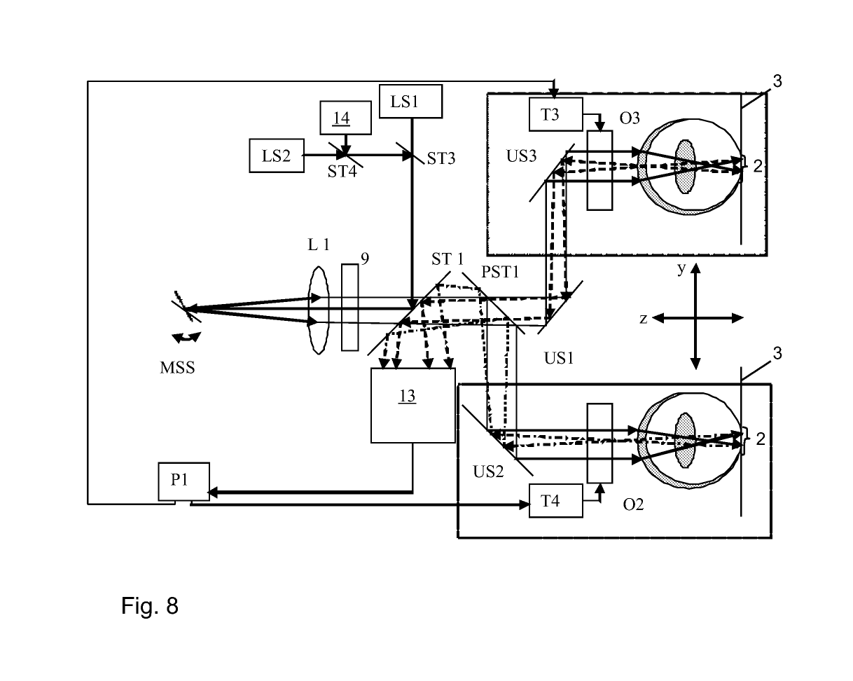

FIG. 8 a schematic block diagram of a projector device as a fourth embodiment of the invention;

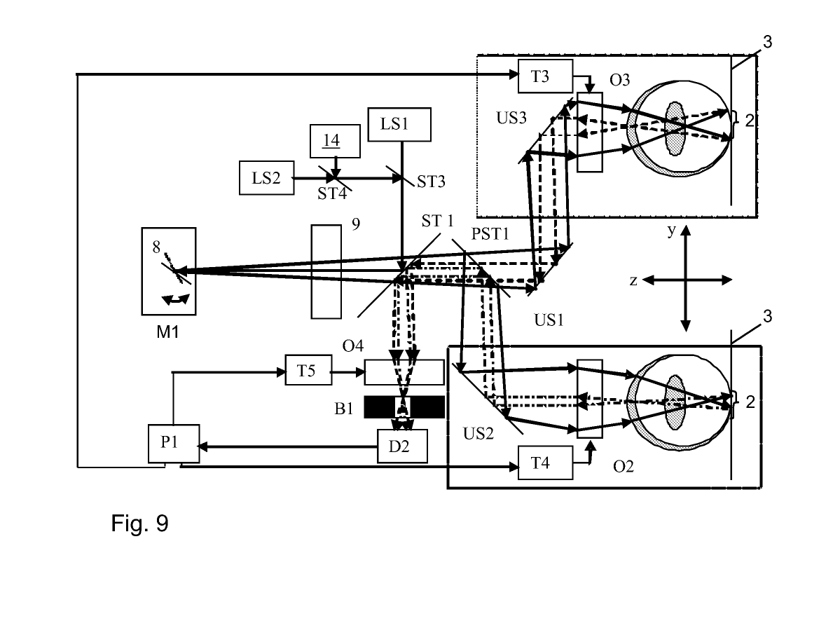

FIG. 9 a schematic block diagram of a projector device as a fifth embodiment of the invention;

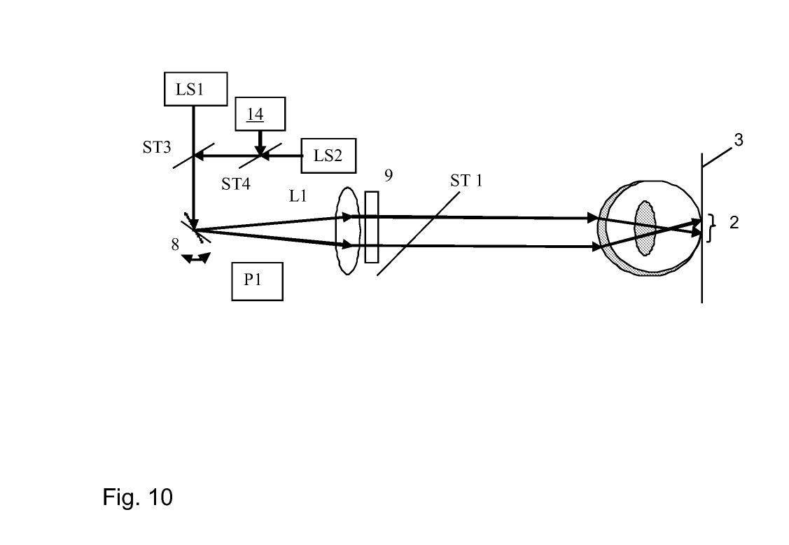

FIG. 10 a schematic block diagram of a projector device as a sixth embodiment of the invention;

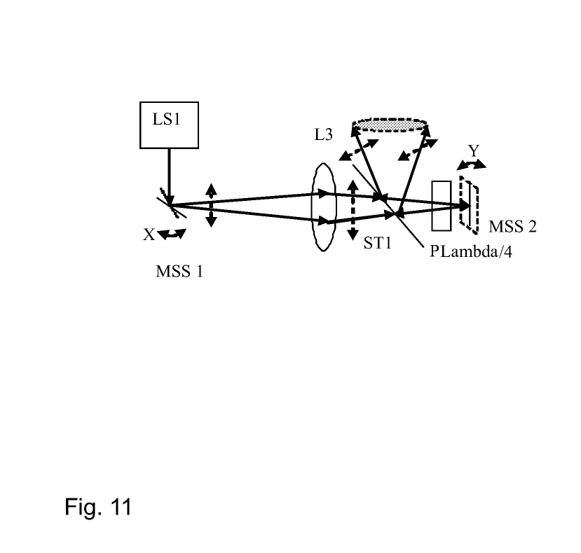

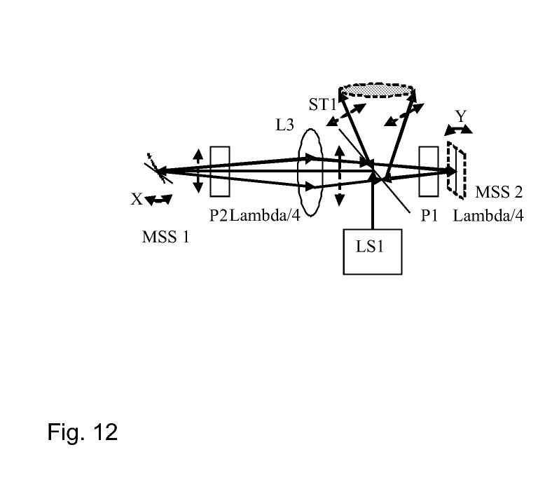

FIGS. 11, 12 possible alternatives to the scanning micromirror shown in the preceding figures.

FIG. 1 provides a very schematic block diagram of a projector device 1 which is designed for projecting a two-dimensional pattern 2 on a plane 3 in a human eye 4. The plane 3 is defined by the area, of the so-called macula inside the retina, which is the area responsible for the visual acuity of the eyes. The two-dimensional pattern 2 may have a size of, for example, 1 mm² in the eye or on the plane 3. The two-dimensional pattern may be designed, for example, as a ring, a geometric figure, a symbol etc. and serves in particular in this embodiment as an eye test character or as an accommodation target for the patient. The accommodation target is projected on the retina and the patient is instructed to focus on it. This brings the viewing direction of the eye and the accommodation of lens 3 in a defined state in order to simplify diagnoses, examinations or treatments. In another embodiment of the projector device 1, the two-dimensional pattern 2 is designed as a measurement area or a measurement pattern and may alternatively be generated, for example, by an invisible light beam. The eye test character or accommodation target may also be colored, in particular multicolored.

The projector device 1 comprises a light source 6 which is designed as a laser beam source and emits a laser beam as a light beam following the bears path 7. The laser beam is linearly polarized. To keep the design size of the projector device 1 small, the diameter of the light, beam is preferably smaller than 0.5 mm.

Following the beam path 7, the light beam first hits a beam splitter ST1, said beam splitter being designed, for example, as polarization-dependent, and is deflected by said beam splitter away from the eye 4 at an angle of about 90 degrees, subsequently, the light beam passes a collimating lens L1 for the first time, then traverses a quarter-lambda waveplate Lambda and subsequently hits a scanning micromirror 8 which can deflect the light, beam in two dimensions at a deflection angle. On the way back the light beam traverses the quarter-lambda waveplate Lambda and the collimating lens L1 again, and is then collimated by said lens. Since passing the quarter-lambda waveplate Lambda twice has changed the polarization, for example from perpendicular polarization to parallel polarization, the light beam can now freely pass the beam splitter ST1, said bears splitter ST1 being designed as polarization-dependent, and subsequently hits an optical module 9, enters the eye 4 and is projected on the plane 3. If the bears splitter ST1 is designed as polarization-independent, the light beam is split. One partial beam then hits the optical module 9 and then enters the eye 9. Since the other partial beam is in this case reflected by the bears splitter ST1 to the laser bears source 6, the polarization-dependent bears splitter ST2 has to reflect the light beam away from the laser source. This protects the laser source. The treatment laser 14 is protected by suitable optical filters and/or optical isolators. By changing the deflection angle for the light beam via the scanning micromirror 8 it is now possible to project the two-dimensional pattern 2 on the plane 3 by writing and scanning, in particular ordered by rows or columns.

The scanning micromirror 8 is designed as an X-Y scanner and has a metallic mirror surface with, a free diameter of, for example, 2 mm and facilitates a deflection of the incident laser beam at a deflection angle in two dimensions with frequencies ranging from 100 Hz to 110 kHz or more. In a particularly simple embodiment of the invention, the scanning micromirror MSS is controlled in a resonance mode so that it always performs the same motion sequence in a reproducible fashion. The motion sequence is chosen such that the laser beam incident at the center of the scanning micromirror 8 is guided by the deflection at the deflection angle such that it traces the surface area of the two-dimensional pattern 2 in a scanning or writing manner. The shape, outline or appearance of the two-dimensional pattern 2 is created by activating or deactivating (more generally: controlling) the light source 6, wherein said light source is activated such that, for example, a ring or a dot is projected as a two-dimensional pattern 2 on the plane 3.

The lens L1 is designed as a collimating lens which aligns the bight beam parallel to the optical axis of the beam path. On its way from the light source 6 to the scanning micromirror 8, the light, beam passes the lens L1 at its center, while on the way from the scanning micromirror 8 to the plane 3, the light beam may traverse the lens L1 outside the center or even in the peripheral region, depending on the deflection angle. In idealized form, the design described so far can generate a sharp two-dimensional pattern 2 on the plane 3, however, the optical system or the projector device 1 and in particular the eye 4 create optical aberrations in the beam path, so that the two-dimensional pattern 2 will only be displayed, out of focus on the plane 3 due to the optical aberrations.

To correct such optical aberrations, the wavefront of the light beam needs to be changed such than each image pixel of the two-dimensional pattern 2 is displayed on the plane 3 such that optical aberrations are corrected, however, this requires that the optical aberrations of the image pixels of the two-dimensional pattern 2 are corrected in a position-resolved way, or in other words, that each light beam leading to an image pixel of the two-dimensional pattern 2 is corrected in its phasing. For this purpose, the optical module 9 is provided which has, when viewed, in the abstract, several regions with different focal lengths. If the two-dimensional pattern 2 is projected on the plane 3, the light source 6 and the scanning micromirror 8 are controlled by a control device 10 such that for each image pixel of the two-dimensional pattern 2 on the plane 3, the light beam is guided through that region of the optical module 9 which is best suited to correct the optical aberration.

To clarify this systematic approach we refer to FIG. 2a which shows the optical module 9 as well as three beam paths a, b, c of a light beam extending from the scanning micromirror 8. In order to simplify the illustration, the collimating lens L1 is not shown. In this example, the optical module 9 has three regions I, II, III which have different focal lengths. An image pixel P located on the optical axis 11 of the design shown is to be generated. If the light beam a is now guided through the region I, the light beam intersects the optical axis 11 at a first position P1; if the light beam b is guided through the region II with a second, longer focal length, the light beam intersects the optical axis 11 at the point P2; if the light beam c is guided through the region III, the light beam intersects the optical axis 11 at the point P3. P1, P2 and P3 are arranged in the axial direction at a distance from each other. By using regions I, II, III with different focal lengths it is thus possible to create an image pixel at different axial positions. The systematic approach explicated above can be used, for example, to compensate for spherical optical aberrations and defocus by guiding the light beam through the correspondingly adapted regions I, II and III.

FIG. 2 b again shows three light beams a, b, c which are, however, all guided through region II, but at different radial positions. Shifting the radial position causes the radial position of the intersection point in plane 3 to shift. It is thus possible to shift the image pixel on the same plane by changing the deflection angle. By shifting all the image pixels it is therefore possible to distort the two-dimensional pattern 2 on the plane 3 in order to compensate for optical aberrations such as coma, astigmatism etc. By using the compensation or correction methods according to FIG. 2 a and FIG. 2 b concurrently it is thus possible to correct any optical aberrations.

FIGS. 3a to 3d are schematic illustrations or different embodiments of the optical module 9. FIGS. 3a and 3b each show a refractive optical element designed as a lens array with different regions 12, wherein the focal lengths of the individual regions 12 differ. FIG. 3c, on the other hand, shows a gradient lens as an optical module 9 in which the different regions 12 are arranged as circular rings. Each of the regions 12 may have a constant focal length, or the focal length may steadily change, for example as a function of the radius. Finally, FIG. 3d shows a diffractive optical element as an optical module 9, again with multiple regions 12, each having a different focal length just as in the preceding figures.

FIG. 1 additionally shows an optional wavefront sensor 13 and an optional treatment laser 14. The wavefront sensor 13 receives the back reflections of the two-dimensional pattern 2 from the eye 4 and uses them to determine the local optical refractive power of the eye 4 and its distribution and thus the optical aberrations in the eye 4. The wavefront sensor is designed, for example, as a Shack-Hartmann sensor or as another aberrometer. The optical aberrations measured are fed into the control device 10, enabling it to initially or steadily improve the display of the two-dimensional pattern 2. The treatment laser 14 is guided via a second beam splitter ST2 along the same axis as the light beam from the light source 6 so that the correction of the optical aberration also improves the beam path of the treatment laser 14, thus achieving improved treatment results.

FIG. 4 provides a very schematic view of a possible embodiment of a portion of the device shown in FIG. 1, illustrating the region of the optical module 9 together with possible embodiments of the beam oath 7 of the accommodation laser beam. The accommodation laser beam is deflected via the scanning micromirror 8 and passes through a center region of the optical module 9 which is designed as a DOE (diffractive optical element). An eye test character is written as a two-dimensional pattern 2 directly and sharply focused on the retina of the eye 4 with the accommodation laser beam. So-called Landolt rings can be produced as eye test characters, for example.

To compensate for any ametropia of the eye 4, the angle of incidence alpha of the accommodation laser beam in the eye 4 can be changed by once again using different radial regions of the DOE 9 with different focal lengths. The DOE 9 is designed, such that the diffraction angle of the DOE 9 is a function of the distance to the optical axis. The aim is to produce an eye test character on the retina of the eye 4 to he measured that is of the same size regardless of the ametropia of said eye. If the ametropia is cylindrical, the eye test character will be projected onto the eye 4 as an ellipse in the corresponding axis of the eye 4 to be measured so that the patient will see a circular ring. If the projector device 1 is integrated into a wave front measuring instrument, said wavefront measuring instrument can be used, to measure and verify the image produced on the retina.

In a similar way as in FIG. 4, FIG. 5 provides a very schematic view of the beam path 7 of the accommodation laser beam with the optical module 9 in the form of a gradient lens. The accommodation laser beam is deflected via the scanning micromirror 8 and passes through a central region of the optical module 9 which is designed as a gradient lens, an eye test character is written directly and sharply focused on the retina of the eye 4 with the accommodation laser beam. To compensate for any ametropia of the eye 4, the angle of incidence alpha of the accommodation laser beam in the eye 4 can be changed by again using different radial regions of the gradient lens, said regions having different focal lengths. The optical module 9 is designed such that the exit angle of the gradient lens is a function of the distance to the optical axis. The aim is to produce an eye test character on the retina of the eye 4 to be measured that is of the same size regardless of the ametropia of said eye.

The illustration in FIG. 6 shows a further embodiment of the invention, wherein the laser bears of a laser bears source LS1 as a light source 6 and the treatment bears of the laser 14 are combined and guided along the same axis via mirrors or beam splitters ST3 and ST4 and then deflected via a deflecting mirror US1 to the scanning micromirror 8. The scanning micromirror 8 deflects the beam path 7 at an angle of 90°. The scanning micromirror 6 and the deflecting mirror US1 are arranged on a slide M1 that can be moved in the direction indicated by arrow A in order to facilitate an adjustment of the distance between the scanning micromirror 8 and the lens L1, so that by moving the slide M1 the local ametropia of the eye can be compensated for by generating additional angular displacements through the scanning micromirror 8 and the upstream optical components L1 and 9.

For the embodiments already illustrated and those described subsequently, the following optional method for setting the measuring distance is proposed: Since the measurement accuracy and the local assignment of the measurement results of a diagnostic device in ophthalmology depends on the distance between the eye 4 and the sensor device 13, the optical module 9 generates a small focal point on the apex of the cornea of the eye that is located exactly at the desired measuring distance. The focal point is generated by guiding the light beam through a measuring point region of the optical module 9 with a corresponding focal length. Said measuring point region may be arranged, for example, in the peripheral area of the optical module. If the eye 4 is not located in the focal point thus generated, a spot of small or large dimensions will appear on the cornea. This spot is evaluated using the observation camera integrated in the sensor device 13 so that the exact measuring distance is shown to the user. The exact measuring distance is reached when the focal point has reached its minimum. The focal point for the measuring distance is used only for the alignment of the projector device 1. For setting the measuring distance, only the projection beans generated by the scanning micromirror 8 are focused by the optical module 9 in a focal point at the desired measuring distance.

An advantage of the embodiments illustrated in FIGS. 6 to 10 and in FIG. 1 is that the focal point for determining the measuring distance is generated by the same light sources 6 and/or LS1 or LS2 which project the two-dimensional patterns 2.

FIG. 7 illustrates a further form of the invention wherein, in addition to the laser beam source LS1, a further laser beam source LS2 is coupled as a light, source 6 into the beam path 7 along the same axis as the laser beam source 14. The laser beams of the two laser beam sources LS1 and LS2 differ in their polarization. The different polarization makes it possible to split the beam path into two different beam paths using a polarization-dependent mirror PST1, so that the first laser beam source LS1 only projects into one eye 4 and the laser beam source LS2 only projects into the other eye 4. This is advantageous because it allows the projector device 1 to provide both eyes 4 simultaneously or in parallel with two-dimensional patterns 2 which may be different, if so desired.

In the constructional embodiment, the laser beams of the laser beam sources LS1 and LS2 and, if applicable, of the treatment laser 14 are guided along the same axis. Behind the beam splitter ST1, the laser beams serving as light beams are again split by the polarization-dependent mirror PST1, according to their polarization, into two separate beam paths. Each beam path is then guided via a deflecting mirror US2 or US3 and an eyepiece O2 or O3 to the respective eye 4. The back reflections of the laser beams and/or the scattered Light from the eyes 4 are again projected onto the censor device 13, so that an actual state of the wavefront of the laser beams emitted by the corresponding laser team source LS1 and LS2 can be recorded for each eye 4 by said sensor device.

The embodiment shown in FIG. 7 is thus an extension of the projector devices 1 illustrated in the preceding FIGS. 1 and 6. This extension facilitates stereoscopic vision, in other words, 3D vision, which can be utilized, for example, to measure ametropia in both eyes under natural conditions. To achieve this, the two-dimensional patterns 2 generated by the scanning micromirror 8 need to be optically separated so that the two-dimensional pattern 2 intended for one eye 4 can only be seen by that eye 4. It is particularly advantageous if each of the laser beam sources is designed as being multicolored so that the two-dimensional patterns 2 are multicolored as well. In this embodiment, multicolored images which show items or objects known to the patient from reality can be used as two-dimensional patterns 2. This helps the patient to perceive the combination of two two-dimensional patterns 2 as one 3D image.

If the two two-dimensional patterns 2 are created by linearly polarized projection beams with different polarization directions which are emitted by the laser beam sources LS1 and LS2, then it is possible to separate the two two-dimensional patterns 2 using the polarization-dependent beam splitter or polarization-dependent mirror PST1. Beams with perpendicular polarization are deflected onto the right eye 4 and beams with parallel polarization are deflected onto the left eye 4 or vice versa. The two two-dimensional patterns 2 are generated simultaneously by modulating the respective laser beam sources LS1 and LS2, which means that the laser beam sources are only activated with respect to the correct color if a corresponding image pixel is to be created on the plane 3. The projector device generates both two-dimensional patterns 2 with the same and maximum possible resolution and the maximum refresh rate, for example, so as to make sure that the image generation of the two-dimensional patterns 2 by scanning the eye 4 is not noticed by the patient.

In another embodiment, it is also possible to separate the two two-dimensional patterns 2 by including polarization-dependent filters in the eyepieces O2 and O3, so that, for example, the right eyepiece O3 can only be passed by beams with perpendicular polarization and the left eyepiece O2 is only transparent to light with parallel polarization. In this case a polarization-independent beam splitter is used instead of the polarization-dependent beam splitter PST1.

In yet another embodiment, the two-dimensional patterns 2 for the right and the left eye 4 may also be projected by a common scanning micromirror 8 in a sequential manner, i.e. in rapid succession, into the eye 4, using just one of the light sources LS1 or LS2. To achieve the 3D effect, shutters would be integrated into both eyepieces O2 and O3. These shutters allow light to pass in turns and only if the respective two-dimensional pattern 2 for the right or the left eye 4 is to be created. The shutters are transparently operated by an evaluation unit P1 synchronously to the image generation for the respective eye 4. The advantage of this embodiment is that only a single light source is needed to generate both two-dimensional patterns 2, said light source being any monochromatic or multicolored light source, in particular an RGB light source. However, while the resolution of the two-dimensional patterns 2 would be sufficient, the refresh, rate would be only half as high as in the example with polarization-dependent laser beam sources LS1 and LS2.

Optionally, the eyepieces O2 and O3 each have at least one lens with electrically controllable focal length. The evaluation unit P1 controls or regulates the focal length of the eyepieces O2 and O3 such that, for example, the mean ametropia, the so-called sphere, of the respective eye 4 is compensated for.

In order to create a sharp image on the retina it is necessary to correct the higher-order optical aberrations such as astigmatism etc. of the respective eye by intelligently controlling the scanning micromirror and the laser beam modulation. Local optical aberrations of the respective eye 4 are corrected by the optical module 9 with location-dependent focal length individually for the respective eye 4, applying the same methods and devices described above in connection with FIG. 1. Error-free imaging makes it possible to perceive a sharp 3D image.

In the embodiment shown in FIG. 7, two supervisory circuits, in particular regulatory or control circuits, can be implemented:

1. Self-correction of the beam path including the eye 4 by determining the actual state of the wavefront of the laser beam from the laser beam source LSI using the sensor device 13, by selectively illuminating the optical module 9 with location-dependent focal length and by controlling the electrically controllable lens in the eyepiece O3.
2. Self-correction of the beam path including the eye 4 by determining the actual state of the wavefront of the laser beam from the laser beam source LS2 using the sensor device W1, by selectively illuminating the optical module 9 with location-dependent focal length and by controlling the electrically controllable lens in the eyepiece O2.

Depending on the design of the polarization-dependent mirror PST1 and of the laser team sources LS1 and LS2, the eyepieces O2 and O3 may be interchanged in the control circuits.

The distance between the eyes of the individual patient must be taken into account when performing binocular measurements. For this reason it is possible to shift the lateral distance between the eyepiece O2, which is rigidly connected to the deflecting mirror US2, and the eyepiece O3, which is rigidly connected to the deflecting mirror US3, in the y direction. In order to achieve a high measurement accuracy, the measuring distance between the eye and tine sensor device 13 designed as a wavefront sensor needs to be kept constant. This means that if the distance between the eyepiece O2 and the eyepiece O3 is decreased, it is necessary to increase the distance between the eyepiece O2 and the eye in the e direction by the same amount, and vice versa. The measuring distance can be checked using the optical component 9, as has been described above. The eyepieces O2 and O3 are always moved by the same distance to the optical axis to ensure that the whole construction remains symmetrical to the optical axis.

FIG. 8 shows a modification of the embodiment shown in FIG. 1 wherein the beam path in front of the scanning micromirror 8 is designed analogously to FIG. 1, so that the description given there can foe referred to.

FIG. 9 shows a possible further form of the sensor device W1 of the preceding figures. In FIG. 9, an exemplary application of tine projector device 1 in a wavefront measuring instrument is illustrated. The eyepieces O2 and O3 compensate for the mean optical aberrations of the eye and the optical module 9 compensates for the higher-order optical aberrations, as described with respect to FIGS. 7 and 8. The scanning micromirror 8, which is mounted on a motorized slide M1 which is movable in the Z direction, is positioned at a specific distance from the eyepieces O2 and O3. The distance from the eyepiece and the adjustable deflection angle of the scanning micromirror 8 ensure that the surface of the eye 4 can be completely scanned on an area of 10×10 mm². With the fixed focal length of the two eyepieces O2 and O3 and the location-dependent focal length of the optical module 9, the laser beams of the laser beam sources LS1 and/or LS2 are refracted at a defined angle, so that an error-free two-dimensional pattern 2 of size 1×1 mm² in particular an image, is displayed sharply on the plane 3 of the retina. The laser beam scattered by the retina of the eye 4 which leaves the eye 4 in close proximity to the apex of the eye 4 is registered by a detector D2. The detector D2 measures the angle of said laser beams and calculates the local ametropia based on the measurement. To ensure the reproducibility of the measurement results and a consistently high measurement accuracy over the entire measurement range, a diaphragm B1 can be displayed exactly on the cornea of the eye 4, preferably within the visual axis of the eye, through the eyepieces O2 and O3 and through the optical component O4 located before the diaphragm B1. The displayed diaphragm B1 guarantees that only those laser beams are evaluated which leave the eye 4 in the vicinity of the apex of the eye 4 through an aperture with a diameter of e.g. 1 mm. Thus only those laser beams are evaluated which are only marginally refracted by the layers of the eye on their way from the plane 3 of the retina to the cornea. In this example, the eyepieces O2 and O3 are designed as optical elements with electrically controllable focal length which are used, for example, to correct the mean optical aberrations of the optical system of the eye 4. The focal length of the eyepieces O2 and O3 is controlled such that the laser beam intersecting the cornea at a particular location enters the eye at an incidence angle that ensures an error-free display on the retina. Changing the focal length of the eyepieces O2 and/or O3 compensates, for example, for the mean ametropia of the eye 4. This change of the focal length of the eyepieces O2 and/or O3, however, causes a change in the location where the diaphragm B1 is displayed, thus distorting the measurement result. This disadvantage is compensated for using the optical component O4 which, like the eyepieces O2 and O3, is designed as an optical component with electrically controllable focal length, by controlling the focal length of the optical component O4 synchronously to the focal length of the eyepiece O2 or O3, depending on which eye 4 is to be measured. In other words, if the focal length f of the eyepiece O2 is changed by ±Δ f, then the focal length of the optical component O4 needs to be changed by the same value ±Δ f at the same time if, for example, the optical design of the eyepieces O2 and O4 is identical.

The eyepieces O2 and O3 can be switched to optically inactive, if required, which means that the evaluation unit P1 sets the longest focal length, if possible 0 dioptres which equals a focal length of ∞ mm. In this case the optical module 9 needs to correct all optical aberrations.

FIG. 10 illustrates at very compact embodiment of a projector device 1. The embodiment can be reduced to the laser beam sources LS1 and, if required, LS2, the scanning micromirror 8, the optical module 9 and the evaluation unit P1. The evaluation unit P1 stores the control signal for the scanning micromirror 8, which was measured during calibration, and the radially location-dependent focal length of the optical module 9. Using these values, the evaluation unit P1 controls the switching on and off of the laser beam, while the scanning micromirror 8 deflects the laser beam so that images are projected free of optical aberrations as two-dimensional patterns 2.

FIG. 11 describes a replacement device for the scanning micromirror 8 of the preceding figures. The objective is to generate the two-dimensional patterns 2 rising individual beams which start from a point source and form a defined angle with the optical axis, so that, for example, a rectangular area can be scanned completely. For this purpose, two one-dimensional scanners MSS1 and MSS2 are utilized. The scanner MSS1 oscillates in the x direction and the scanner MSS2 oscillates in the y direction. The laser beam emitted by the laser beam source LSI first hits, for example, the scanner MSS1 which oscillates in the x direction. Said scanner deflects the projection beam at an angle α in the x direction. To achieve the advantages of a point source, or to replicate the advantages of a 2D scanning micromirror, the laser beam reflected by the scanner MSS1 is focused onto the one-dimensional scanner MSS2 by the lens L3 which, for example, may be designed as an asphere. The lens L3 may also be designed as a functionally equivalent objective. The scanner MSS2 oscillates in the y direction and additionally deflects the projection beam at an angle β in the Y direction. The projection beam thus receives a deflection in the x and y directions and fully replaces a 2D scanning micromirror. If the scanners MSS1 and MSS2 are not designed as resonance oscillators but as galvano scanners then each point of the projection surface can be controlled at any given time and for as long as the application requires.

To prevent multiple reflections between the scanning mirrors MSS1 and MSS2, the light source LS1 is designed as a linearly polarized light source. The polarization-dependent beam splitter ST1 lets one polarization direction pass and reflects the other polarization direction. To ensure that the beam bundle is not reflected back onto the scanner MSS1 by the scantier MSS2, the polarization direction of the projection beam is changed by the λ/4 plate P Lambda/4. On the way to the scanner MSS2, the λ/4 plate P Lambda/4 generates right circularly polarized light which is reflected by the scanner MSS2, which is designed as a metallic mirror. On the way from the scanner MSS2 to the bears splitter ST1, the light, which is now left circularly polarized because of the reflection at the metallic mirror, is transformed by the λ/4 plate P Lambda/4 into linearly polarized light. The polarization direction thus rotates from, for example, perpendicular to parallel or vice versa.

FIG. 12 illustrates a further replacement device for the scanning micromirror 8. The laser beam LS1 with e.g. perpendicular polarization is reflected by the polarization-dependent beam splitter ST1 onto the scanning micromirror MSS1. After two passes, the λ/4 plate P1 Lambda/4 has rotated the polarization direction from e.g. perpendicular to parallel, so that the beam splitter ST1 lets the projection beam, which has been focused by the lens L3, pass to the 1D scanning micromirror MSS2.

The focal point lies on the scanning micromirror MSS2. The λ/4 plate P2 Lambda/4 in conjunction with the scanning micromirror MSS2, which is designed as a metallic mirror, rotate the polarization direction from e.g. parallel to perpendicular, so that the projection beam reflected by the scanning micromirror MSS2 is reflected in two dimensions in an upward direction by the beam splitter ST1.

The λ/4 plate P1 Lambda/4 or P2 Lambda/4 generates circularly polarized light from a linearly polarized projection beam and linearly polarized light from circularly polarized light. In doing this it rotates the polarization direction of the linearly polarized projection beam from e.g. perpendicular polarization to parallel polarization or vice versa, since the non-depolarizing scanning micromirror MSS1 or MSSS2 generates e.g. left circularly polarized light from right circularly polarized light by reflecting it.

### REFERENCE SYMBOL LIST

1 projector device
2 pattern
3 plane
4 eye
5 lens
6 light source
7 beam path
8 scanning micromirror
9 optical module
10 control device
11 optical axis
12 regions
13 wavefront sensor
14 treatment laser
ST1 beam splitter
ST2 beam splitter
lambda lambda/4 plate
B1 diaphragm
W1 wavefront measuring instrument/ametropia measuring instrument
LS1 projection beam; source (RGB laser diode or SLD and IR), parallel polarization
LS2 projection beam source (RGB laser diode or SLD and IR), perpendicular polarization
BL treatment laser
L1 lens (glass or plastic lens)
L3 lens (glass or plastic lens)
MSS 2D micro-scanning mirror
MSS1 1D micro-scanning mirror
MSS2 1D micro-scanning mirror
PST1 polarization-dependent beam splitter
ST1 beam splitter
ST2 beam splatter
ST3 beam splitter
ST4 beam splitter
US1 deflecting mirror
US2 deflecting mirror
US3 deflecting mirror
P Lambda/4 λ/4 plate
P1 Lambda/4 λ/4 plate
P2 Lambda/4 λ/4 plate
O2 eyepiece with electrically controllable focal length and/or shutter
O3 eyepiece with electrically controllable focal length and/or shutter
O4 objective with electrically controllable focal length
D2 detector (PSD or CCD/CMOS camera)
P1 digital processor/control
T3 driver for the electrically controllable eyepieces
T4 driver for the electrically controllable eyepieces
T5 driver for the electrically controllable eyepieces

1. A projector device for projecting a two-dimensional pattern on a plane in an optical body, in particular in an eye,
with a light source which is designed to emit a light beam,
with a deflecting device which is designed to deflect the light beam in order to generate the two-dimensional pattern,
with an optical module which is arranged between the light source and the plane,
with a control device which is designed to control the deflecting device such that the two-dimensional pattern is formed on the plane,
wherein:
the optical module has at least two regions with different focal lengths through which said light beam can be guided in an alternating or consecutive manner by means of the deflecting device, and the control device is designed to control the deflecting device and the light source such that the light beam is guided through the at least two regions in order to compensate for position-resolved aberrations of the optical body and to project the two-dimensional pattern on the plane such that optical aberrations are corrected.

2. The projector device of claim 1, wherein the light beam is guided through different regions of the optical module with different focal lengths for different image pixels of the two-dimensional pattern.

3. The projector device of claim 1, wherein the two-dimensional pattern is designed as an accommodation target or as an eye test character for the eye,

4. The projector device of claim 1, wherein the light source is designed as a laser beam source which facilitates the emission of several laser beams of different colors such that the two-dimensional pattern appears multicolored.

5. The projector device of claim 1, wherein the light beam is invisible.

6. The projector device of claim 1, wherein the two-dimensional pattern is designed as a measurement pattern,

7. The projector device of claim 1, wherein the control device is designed such that it can move the two-dimensional pattern within the plane.

8. The projector device of claim 1, wherein the control device is designed such that it can control the deflecting device with always the same deflection pattern or with any irregular deflection pattern, and such that it can control the selection of the regions of the optical module and the shaping of the two-dimensional pattern by switching the light source on and off.

9. The projector device of claim 1, wherein the deflecting device is designed as a 2D deflecting device and comprises two 1D deflecting devices (MSS1, MSS2) arranged separately to each other.

10. The projector device of claim 1, wherein the optical module has several separate regions with different focal lengths and/or transition zones between the at least two regions in which the focal lengths are steadily changing.

11. The projector device of claim 1, wherein the optical module has a region designed as a measuring point region, wherein the focal length of the measuring point region is chosen such that the focal point is created on the cornea of the eye, and wherein the projector device is designed to check for the correct distance between the projector device and the eye based on the focal point on the cornea.

12. The projector device of claim 1, wherein the optical module is designed as a diffractive optical module.

13. The projector device of claim 1, wherein the optical module is designed as a refractive optical module.

14. The projector device of claim 1, wherein the optical module is designed as a reflective optical module.

15. The projector device of claim 1, wherein the optical module is designed as a holographic optical module.

16. The projector device f claim 1, wherein the projector device is designed to simultaneously project two-dimensional patterns into both eyes of a patient and/or designed as a binocular or stereoscopic device.

17. A medical device with the projector device of claim 1, wherein the medical device has a wavefront sensor for determining the position-resolved aberrations of the optical body, in particular the eye.

18. The medical device of claim 17, wherein the control device is designed, due to its programming and/or wiring, such that a control circuit is established, wherein the deflecting device and the light source are controlled, based on the position-resolved optical aberrations that have been determined, in such a way that the two-dimensional pattern is projected on the plane such that optical aberrations are corrected.

19. The medical device of claim 17, wherein layers are arranged within the optical body, in particular the eye, wherein the medical device, in particular the projector device, evaluates exactly or less than the back reflection from the layers.

20. The medical device of claim 17, wherein a treatment laser for the treatment of the optical body, in particular the eye, wherein the laser beam of the treatment laser is guided along the same axis as the light beam.
